(19) United States
(12) Patent Application Publication
(10) Pub. No.: US 2019/0183817 A1
(43) Pub. Date: Jun. 20, 2019

(54) EXTENDED RELEASE SUSPENSIONS OF MIXTURES OF DEXTRO- AND LEVO-AMPHETAMINES

(71) Applicant: LUPIN INC., Somerset, NJ (US)

(72) Inventors: Bharat Kirthivasan, Somerset, NJ (US); Sabarinath Shankar, Somerset, NJ (US); James Garegnani, Somerset, NJ (US); Arjun Muthukkaruppan, Somerset, NJ (US)

(21) Appl. No.: 16/214,336

(22) Filed: Dec. 10, 2018

Related U.S. Application Data
(60) Provisional application No. 62/599,357, filed on Dec. 15, 2017.

(51) Int. Cl. A61K 31/137; A61K 9/50; A61K 47/58

(52) U.S. Cl. CPC A61K 31/137; A61K 9/5084; A61K 47/58; A61K 9/5047

(57) ABSTRACT

The present invention relates to the pharmaceutical compositions of a mixture of dextro- and levo-amphetamines complexed with ion exchange resin and precursor resins. More particularly the invention relates to Attention Deficit Hyperactivity Disorder (ADHD) effective agent dosage forms that both facilitate oral ingestion and provide an effective treatment over a prolonged period oftime. In particular, the invention provides for pharmaceutical compositions having a first and second plurality of particles, where the first plurality of particles comprises drug-resin particles and the second plurality of particles comprises drug-precursor resin particles with or without any extended release coating, where the drug is an ADHD effective agent and the composition achieves an escalating in vivo plasma concentration of the ADHD effective agents selected from amphetamine and methylphenidate.

### FIELD OF INVENTION

The present invention relates to the pharmaceutical compositions of a mixture of dextro- and levo-amphetamines complexed with ion exchange resin and precursor resins. More preferably, the invention relates compositions comprising mixture of dextro- and levo-amphetamines administered through oral route in the form of tablets, suspensions, orally disintegrating tablets. The invention also relates to methods of making such compositions and methods of treating using such composition.

The invention relates to Attention Deficit Hyperactivity Disorder (ADHD) effective agent dosage forms that both facilitate oral ingestion and provide an effective treatment over a prolonged period of time. In particular, the invention provides for pharmaceutical compositions having a first and second plurality of particles, where the first plurality of particles comprises drug-resin particles and the second plurality of particles comprises drug-precursor resin particles with or without any extended release coating, where the drug is an ADHD effective agent and the composition achieves an escalating in vivo plasma concentration of the ADHD effective agents selected from amphetamine and methylphenidate.

### BACKGROUND OF THE INVENTION

Many drug therapies use immediate-release oral dosage forms administered at spaced intervals to provide and maintain a desired therapeutic effect over a prolonged therapy period. For example, drugs used in treating Attention Deficit Disorder (ADD) and ADHD such as ADDERALL® and RITALIN® are administered two or three times a day.

For various reasons, subjects often experience difficulty complying with this administration schedule. In particular, because ADD and ADHD are commonly diagnosed in children, the dosage regimen generally requires that at least one dose is administered during the school day. Children are typically not permitted to self-administer the drug at school. As such, authorized school personnel generally take on the responsibility for administering the drug to children during the school day. However, this approach raises issues of medical privacy and potential stigmatizing of the child by peers. In addition, the compliance issue becomes further complicated as transportation, storage and supply of the drug typically must be documented and/or monitored, and the schedules of the different parties involved, i.e., the child, the educators and the authorized school personnel, must be coordinated and accommodated. The unfortunate result is that doses may be given late or missed altogether resulting in decreased efficacy of the therapy.

To avoid administering multiple doses during the day, once-a-day sustained and extended release medications have been developed. For example, ADDERALL XR®, a mixed amphetamine salts medication, is administered once-a-day for the treatment of ADD and ADHD. To achieve extended release, ADDERALL XR requires a mixture of four amphetamine salts: dextro-amphetamine sulfate, dextro-amphetamine saccharate, amphetamine aspartate, and amphetamine sulfate.

METADATE CD® is another once-a-day ADHD treatment. This formulation comprises methylphenidate hydrochloride and achieves an extended-release profile through its makeup of 30% immediate release beads and 70% extended release beads.

Prior art ADHD compositions such as ADDERALL XR and METADATE CD are only available in solid dosage forms. Many people, especially children, have difficulty swallowing standard solid dosage forms. Accordingly, there is a need in the art to develop easily ingested, once-daily oral compositions that provide effective, prolonged treatment.

U.S. Pat. Nos. 9,017,731 and 8,709,491 which disclose pharmaceutical composition comprising mixtures of dextro- and levo-amphetamines complexed with ion-exchange resin particles wherein 30-50% by weight of said amphetamines are present in a first plurality of uncoated drug-resin particles and 50% to 70% by weight of said amphetamines are present in second plurality of drug-resin particles that are coated with delayed release coating.

Although prior art discloses various attempts to prepare the compositions of amphetamines, there is still a need for once-a-day formulation that is easily ingested, without going through extensive, lengthy coating manufacturing process, of the fine resin particles.

### DETAILED DESCRIPTION OF INVENTION:

In main object of the invention to provide pharmaceutical compositions of a mixture of dextro- and levo-amphetamines complexed with ion exchange resin and precursor resins. In particular, the invention provides for easily ingested, once-daily pharmaceutical compositions that provide effective, prolonged treatment.

In another embodiment, the invention provides various advantage over prior art compositions and methods. For example, the invention provides for liquid drug suspensions which are favored by individuals who have difficulty swallowing conventional solid dosage forms (e.g., children or dysphagic individuals). Moreover, the compositions comprise ion-exchange resins and thus have enhanced taste masking properties as compared to traditional drug formulations. Also the invention have the advantage of eliminating the complex and lengthy coating process involved in fine resin particles coating.

In particular, the invention relates to pharmaceutical compositions comprising a plurality of particles, wherein the drug in said drug-resin particles comprises mixture of dextro- and levo-amphetamines. For example, in one embodiment, the pharmaceutical composition comprises (a) mixture of dextro- and levo-amphetamines-resin complex providing for immediate release; and (b) mixture of dextro- and levo-amphetamines-precursor resin complex covered with or without an extended release coating, wherein said component (a) provides for an immediate release of amphetamines from the drug resin complex to provide a first blood level of amphetamines and component (b) provides an extended release of amphetamines from the drug-precursor resin complex that increases the blood level of amphetamine to a second level.

In one embodiment, the pharmaceutical compositions of invention comprises amphetamines such as a mixture of amphetamine and dextro-amphetamine (e.g., a mixture of 75% dextro-amphetamine and 25% levo-amphetamine). In one embodiment, the compositions are substantially free of dextro-amphetamine saccharate and/or amphetamine aspartate.

In another embodiment, the compositions comprise a first plurality of drug-resin particles that are not coated with an extended release coating, and a second plurality of drug-precursor resin particles that are coated with an extended release coating. The extended release may comprise diffusion barrier coating (e.g., a water insoluble, water permeable membrane such as ethylcellulose).

In another embodiment, the resin particles are strong acidic cation exchange resins such as polistirex, polacrilex, or polacrilin. In other embodiments, the resin particles are AMBERLITE® IRP64, IRP69, or IRP88 resins, or DUOLITE® AP143 resins. Preferably AMBERLITE® IRP69 having particle size in the range of 25 microns to150 micron, more preferably the average particle size of d (50)=50 micron.

In another embodiment, the precursor resins are ion exchange resins having particle size greater than 250 micron. In another embodiment, the precursor resins having particle size in the range of 250 microns to 1180 micron. More preferably the average particle diameter for precursor resin is d (50)=425 micron. The precursor resins are the whole bead form of sodium polystyrene sulfonate (AMBERLITE IRT69) resins. In one embodiment, a pharmaceutical composition comprising a mixture of dextro- and levo-amphetamines complexed with ion-exchange resin particles to form drug resin particles, wherein 5% to 50% by weight of said amphetamines are present in a first plurality of drug-resin particles that are uncoated and 50% to 95% by weight of said amphetamines are present in a second plurality of drug-precursor resin particles.

In another embodiment, a pharmaceutical composition comprising a mixture of dextro- and levo-amphetamines complexed with ion-exchange resin particles to form drug resin particles, wherein 10% to 40% by weight of said amphetamines are present in a first plurality of drug-resin particles that are uncoated and 60% to 90% by weight of said amphetamines are present in a second plurality of drug-precursor resin particles that are coated with an extended release coating.

In another embodiment, a pharmaceutical composition comprising a mixture of dextro- and levo-amphetamines complexed with ion-exchange resin particles to form drug resin particles, wherein 20% to 30% by weight of said amphetamines are present in a first plurality of drug-resin particles that are uncoated and 70% to 80% by weight of said amphetamines are present in a second plurality of drug-precursor resin particles that are coated with an extended release coating.

Ion-exchange resins suitable for use in the preparations and methods described herein are water-insoluble and comprise an indigestible organic and/or inorganic matrix containing covalently bound functional groups that are ionic or capable of being ionized under the appropriate conditions of pH. The organic matrix may be synthetic (e.g., polymers or copolymers of acrylic acid, methacrylic acid, sulfonated styrene, sulfonated divinylbenzene), or partially synthetic (e.g., modified cellulose and dextrans). The inorganic matrix preferably comprises silica gel modified by the addition of ionic groups. Covalently bound ionic groups may be strongly acidic (e.g., sulfonic acid, phosphoric acid), weakly acidic (e.g., carboxylic acid), strongly basic (e.g., primary amine), weakly basic (e.g. quaternary ammonium), or a combination of acidic and basic groups. In general, the types of ion-exchangers suitable for use in ion-exchange chromatography and for such applications as deionization of water are suitable for use in the controlled release of drug preparations. Suitable ion exchange resins are also sold under the trade names AMBERLITE and Dowex. Such ion-exchangers are described by H. F. Walton in “Principles of Ion Exchange” (pp. 312-343) and “Techniques and Applications of Ion-Exchange Chromatography” (pp. 344-361) in Chromatography. (E. Heftmann, editor), Van Nostrand Reinhold Company, New York (1975), incorporated herein by reference. Exemplary ion-exchange resins that can be used in the present invention have exchange capacities below about 6 milli equivalents (mEq)/gram and preferably below about 5.5 mEq/gram.

The rate of ion-exchange reactions depends on the size of the resin particles. Decreasing the size of resin particles significantly decreases the time required for the reaction to reach equilibrium with the surrounding medium and vice versa.

Typically, the size of the ion-exchange particles is from about 25 microns to about 600 microns. Particle sizes substantially below the lower limit are difficult to handle in all steps of the processing. Both regularly and irregularly shaped particles may be used as resins. Regularly shaped particles are those particles that substantially conform to geometric shapes, such as spherical, elliptical, cylindrical and the like, which are exemplified by Dow XYS-40010.00 and Dow XYS-40013.00 (The Dow Chemical Company). Irregularly shaped particles are all particles not considered to be regularly shaped, such as particles with amorphous shapes and particles with increased surface areas due to surface channels or distortions. Irregularly shaped ion-exchange resins of this type are exemplified by AMBERLITE IRP-69 (Rohm and Haas). Two of the preferred resins of this invention are AMBERLITE IRP-69 (Colorcon) and Purolite C100MRNS (Purolite). Both are sulfonated polymers composed of polystyrene cross-linked with 8% of divinylbenzene, with an ion-exchange capacity of about 4.5 to 5.5 mEq/g of dry resin. Their essential difference is in physical form. AMBERLITE IRP-69 consists of irregularly-shaped particles with a size range of 47 micron to 149 micron produced by milling the parent large-sized spheres of AMBERLITE® IRP-120. The Dow XYS-40010.00 product consists of spherical particles with a size range of 45 micron to 150 micron. Other useful resins include, AMBERLITE IR69F, the Sodium polystyrene sulfonate spherical beads, which are generally referred as the Precursor beads of AMBERLITE IRP69 resin. The typical particle size of AMBERLITE IR69F beads range between 250-600 microns; AMBERLITE IRP476, the Sodium polystyrene sulfonate resins with particle size between 75-150 microns, d(50)-125 microns; AMBERLITE IRP469, another Sodium polystyrene sulfonate resin with particle size between 50-150 microns, d(50)-100 microns.

Binding of drug to resin can be accomplished using methods known in the art. Indeed, one of ordinary skill in the art can easily determine the appropriate method depending upon the drug. Typically four general reactions are used for a basic drug, these are: (a) resin (Na+-form) plus drug (salt form); (b) resin (Na+-form) plus drug (as free base); (c) resin (H+-form) plus drug (salt form); and (d) resin (H+-form) plus drug (as free base). All of these reactions except (d) have cationic by-products and these by-products, by competing with the cationic drug for binding sites on the resin, reduce the amount of drug bound at equilibrium. For basic drugs, stoichiometric binding of drug to resin is accomplished only through reaction (d).

Typically, the ion-exchange resin, in the form indicated by the chosen reaction, is placed in an aqueous solution of the chosen form of drug and agitated. The drug-resin complex thus formed is collected and washed with deionized or purified water to ensure removal of any unbound drug. The complexes are then dried.

Uncoated drug-resin complexes rapidly release the drug in the subject, such as, for example, in the gastrointestinal tract. To delay the release of drug from the drug-resin complex, the complex may be coated as described below.

The amount of drug that can be loaded onto a resin will typically range from about 1% to about 80%, preferably about 15% to about 60%, by weight of the loaded drug-resin particles. A skilled artisan with little or no experimentation can readily determine the optimum loading for any drug resin complex. In a preferred embodiment, loadings of about 30% to about 60% by weight of the drug-resin particles can be employed.

Those of skill in the art will appreciate that certain drugs will have an affinity for particular types of resins.

The inventors have determined that the loading levels of amphetamine are suitable on strongly acidic cation exchange resins such as AMBERLITE IRP-64 and AMBERLITE IRP-69 resins. Resins useful in the invention with modification of the API are strong base anion exchange resins (e.g., DUOLITE.™. AP143) and weak acid cation exchange resins (e.g., AMBERLITE IRP-88).

Amphetamine resin complexes can be formed using any amphetamine salt, since the salt counter-ion is replaced by the ion exchange resin, and release of the drug is controlled by coating and ionic bonding, rather than differential solubility of the salts. In a preferred mode, resin particles are loaded using a single salt of racemic amphetamine and a single salt of dextroamphetamine.

Drug-precursor resin particles may be coated with an extended release coating comprising a water-permeable, film-forming polymer. Any coating procedure which provides a contiguous coating on each particle of drug-resin complex without significant agglomeration of particles may be used. Coatings may be applied with a fluid-bed coating apparatus having the Wurster configuration. Measurements of particle size distribution can be done before and after coating to show that agglomeration of particles is acceptable.

The polymer may be any of a large number of natural or synthetic film-formers used singly, or in admixture with each other, and optionally in admixture with plasticizers, pigments and other substances to alter the characteristics of the coating. In general, the extended release coating should be insoluble or slowly soluble in water and permeable to water. Additional examples of coating polymers are described by R. C. Rowe in Materials Used in Pharmaceutical Formulation (A. T. Florence, editor), Blackwell Scientific Publications, Oxford, 1-36 (1984), incorporated by reference herein.

Preferably, the extended release coating comprises ethyl cellulose, for example, an ethyl cellulose having the content of ethoxyl group from 44 to 47.5%, preferably from 45 to 46.5%. In embodiments of the present invention, the inclusion of an effective amount of a plasticizer in the aqueous dispersion of hydrophobic polymer will further improve the physical properties of the film. For example, because ethylcellulose has a relatively high glass transition temperature and does not form flexible films under normal coating conditions, it may be necessary to add plasticizer to the ethylcellulose before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after routine experimentation with the particular coating solution and method of application.

Examples of suitable plasticizers for ethylcellulose include water insoluble plasticizers such a dibutyl sebacate, diethyl phthalate, triethyl citrate, tributyl citrate and triacetin, although it is possible that other water-insoluble plasticizers (such as acetylated monoglycerides, phthalate esters, castor oil, etc.) may be used. A plasticizer such as Durkex 500 vegetable oil may also be incorporated to improve the film forming property. In one alternative, it is desirable to incorporate a water-soluble substance, such as methyl cellulose, to alter the permeability of the coating.

The extended release coating materials can be applied as an aqueous suspension. One commercially available aqueous dispersion of ethylcellulose is Aquacoat® (FMC Corp., Philadelphia, Pa., U.S.A.). Aquacoat® is typically prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudo latex. The plasticizer is not incorporated in the pseudo latex during the manufacturing phase. Thus, prior to using the same as a coating, it is preferable to intimately mix the Aquacoat® with a suitable plasticizer prior to use.

Another aqueous dispersion of ethylcellulose is commercially available as Surelease® (Colorcon, Inc., West Point, Pa., U.S.A.). This product is typically prepared by incorporating plasticizer into the dispersion during the manufacturing process. A hot melt of a polymer, plasticizer (e.g., dibutyl sebacate), and stabilizer (e.g., oleic acid) may be prepared as a homogeneous mixture, which is then diluted with an alkaline solution to obtain an aqueous dispersion which can be applied directly onto substrates.

Another alternative coating material is a readily available Opadry EC—a fully formulated ethylcellulose product, with plasticizer, and with and without pore-formers. Available in 3 forms, without any pore former, with low and high pore former.

Another alternative coating material is a mixture of an insoluble, film-forming polymer and a water soluble pore former or polymer, i.e., refers to a two-component system. One preferred water soluble polymer is methyl cellulose, which may be used in a two-component system with ethylcellulose.

Another alternative coating material is a mixture of two insoluble, film-forming polymers; for example polyvinyl acetate phthalate (PVAP) and ethylcellulose. Another alternative coating material is polyvinyl pyrrolidone (PVP), polyvinyl alcohol, polyvinyl acetate and mixtures thereof.

Typically, the water-permeable, film-forming polymer comprises from about 1% to about 60% by weight of the drug-resin complex, and preferably from about 20% to about 50% by weight of the dry resin. In terms of coat thickness, preferably, the diffusion barrier coat thickness is at least 5 microns and more preferably, the diffusion barrier coat thickness is from about 10 microns to about 50 microns. Optimum coat weight and coat thickness may be determined for each drug-resin complex and generally depend on the drug release characteristics of the resin for a particular drug. For example, to achieve drug release times within about 1 hour to about 4 hours, the drug-resin complex may be coated with a light coat weight. A light coat weight is a coat weight present in the amount of about 10% to about 20% by weight of the dry resin. To achieve drug release times from about 6 hours to 10 hours, a medium coat weight may be used, i.e. a coat weight present in the amount of 30% to about 35% by weight. To achieve drug release times for about 12 hours, a heavy coat weight may be used, i.e. a coat weight of about 40% to 50% by weight of the dry resin.

In yet another embodiment, the invention provides for liquid oral dosage forms, orally disintegrating tablets, sublingual tablets, and/ or buccal tablets. These dosage forms have distinct advantages over prior art solid dosage forms including dosage flexibility and ease of swallowing. Liquid dosage forms are especially preferred for pediatric use.

Liquid oral dosage forms include aqueous and non-aqueous solutions, emulsions, suspensions, and solutions and/or suspensions reconstituted from non-effervescent granules, containing suitable solvents, preservatives, emulsifying agents, suspending agents, diluents, sweeteners, coloring agents, and flavoring agents. Liquid forms, such as syrups and suspensions, preferably contain from about 1% to about 50%, and more preferably from about 1% to about 25%, and most preferably from about 3% to about 10%, of the drug-resin complex. Other optional ingredients well known to the pharmacist's art may also be included in amounts generally known for these ingredients, for example, natural or artificial sweeteners, flavoring agents, colorants and the like to provide a palatable and pleasant looking final product; acidulants, for example, citric acid, ascorbic acid, or malic acid and the like to adjust pH; antioxidants, for example, butylated hydroxy anisole or butylated hydroxy toluene; and preservatives, for example, methyl or propyl paraben or sodium benzoate, to prolong and enhance shelf life.

In preparing the liquid oral dosage forms, the drug-resin complexes and drug-precursor resin complexes are incorporated into an aqueous-based orally acceptable pharmaceutical carrier consistent with conventional pharmaceutical practices. An “aqueous-based orally acceptable pharmaceutical carrier” is one wherein the entire or predominant solvent content is water. Typical carriers include simple aqueous solutions, syrups, dispersions and suspensions, and aqueous based emulsions such as the oil-in-water type. Preferably, the carrier is a suspension of the pharmaceutical composition in an aqueous vehicle containing a suitable suspending agent. Suitable suspending agents include Avicel RC-591 (a microcrystalline cellulose/sodium carboxymethyl cellulose mixture available from FMC), guar gum and the like. Such suspending agents are well known to those skilled in the art. While the amount of water in the compositions of this invention can vary over quite a wide range depending upon the total weight and volume of the drug-resin complex and other optional non-active ingredients, the total water content, based on the weight of the final composition, will generally range from about 20 to about 75%, and, preferably, from about 20 to about 40%, by weight/volume.

Although water itself may make up the entire carrier, typical liquid formulations may contain a co-solvent, for example, propylene glycol, glycerin, sorbitol solution and the like, to assist solubilization and incorporation of water-insoluble ingredients, such as flavoring oils and the like, into the composition. In general, therefore, the compositions of this embodiment preferably contain from about 5 to about 25 volume/volume percent and, most preferably, from about 10 to about 20 volume/volume percent, of the co-solvent.

In another embodiment, a pharmaceutical composition is liquid oral dosage form. In preferred embodiment pharmaceutical compositions of invention are suspension.

In another embodiment, a pharmaceutical composition comprising a mixture of dextro- and levo-amphetamines complexed with ion-exchange resin particles to form drug resin particles, wherein 20% to 30% by weight of said amphetamines are present in a first plurality of drug-resin particles that are uncoated and 70% to 80% by weight of said amphetamines are present in a second plurality of drug-precursor resin particles that are coated with extended release coating comprising ethyl cellulose.

In another embodiment, a pharmaceutical composition comprising a mixture of dextro- and levo-amphetamines complexed with ion-exchange resin particles to form drug resin particles, wherein 1% to 30% by weight of said amphetamines are present in a first plurality of drug-resin particles that are uncoated and 70% to 99% by weight of said amphetamines are present in a second plurality of drug-precursor resin particles that may or may not be coated with extended release coating comprising ethyl cellulose.

In another embodiment, a pharmaceutical composition comprising a mixture of dextro- and levo-amphetamines complexed with ion-exchange resin particles to form drug resin particles; wherein the composition comprises two drug-resin complexes; (a) mixture of dextro- and levo-amphetamines-resin complex providing for immediate release; and (b) mixture of dextro- and levo-amphetamines-precursor resin complex covered with an extended release coating; wherein 1% to 30% by weight of the amphetamines are present in (a) drug-resin complex and 70% to 99% by weight of the amphetamines are present in (b) drug resin complex.

In another embodiment, a pharmaceutical composition comprises extended release coating comprising water permeable and film forming polymer. More preferably, ethyl cellulose.

In another embodiment, a pharmaceutical composition comprises (a) drug resin complex comprises mixture of dextro- and levo-amphetamines and sodium polystyrene sulfonate having particle size in the range of 47 to 149 microns.

In another embodiment, a pharmaceutical composition comprises (b) drug resin complex comprises mixture of dextro- and levo-amphetamines and sodium polystyrene sulfonate beads having particle size in the range of 250 to 600 microns.

In another embodiment, a method of preparing pharmaceutical liquid compositions in the form of suspension.

The invention also provides for methods of treating various conditions such as Attention-Deficit Disorder or ADHD, fatigue, obesity or imparting alertness, by administering an effective amount of the compositions described herein. In one embodiment, the amount of drug delivered to the subject is between about 2 mg/24 hours to about 60 mg/24 hours. In another embodiment, the amount of drug delivered to the subject is about 5 mg/24 hours to about 30 mg/24 hours. In particular embodiments, the effective amount is 0.5 mg/kg/day to 1.5 mg/kg/day, 0.25 mg/kg/day to 0.5 mg/kg/day, or 0.28/kg/day to 0.4 mg/kg/day. The composition may be administered once-a-day as a single or multiple unit dose. This invention is preferred for a subject suffering from dysphagia.

Many other variations of the present invention will be apparent to those skilled in the art and are meant to be within the scope of the claims appended hereto. The foregoing specification alludes to beliefs, hypothesis and conclusions of the inventors based on his experience in the field, the reports of others and experiments conducted and reported herein, and are provided for purposes of (possible) explanation only and are not meant to limit the invention in any manner whatsoever.

The following examples illustrate various aspects of the present invention, and are set forth to assist in understanding the invention. These examples should not be construed as specifically limiting the invention described and claimed herein. Variations of the invention, including the substitution of all equivalents now known or later developed, which would be within the purview of those skilled in the art, and changes in formulation or minor changes in experimental design, are considered to fall within the scope of the invention and appended claims.

### EXAMPLES

### Example 1

### Composition (80: 20% of Drug-Precursor Resin Complex: Drug-Resin Complex)

| | | | | Amount/ |
|---|---|---|---|---|
| Sr. | | | Amount | Unit |
| No. | Ingredients | | (%) | (mg) |
| 1 | Drug - Precursor Resin Complex | | | 45.12 |
| | a. | Amphetamine sulfate (as base) | 13.33 | 7.52 |
| | b. | Dextroamphetamine sulfate | 13.33 | 7.52 |
| | | (as base) | | |
| | c. | Sodium Polystyrene Sulfonate | 53.34 | 30.08 |
| | | (Amberlite IR69F) (dried basis) | | |
| | d. | Purified Water^{&} | — | Q.S |
| 2. | Drug - Resin Complex | | | 11.28 |
| | e. | Amphetamine sulfate (as base) | 3.33 | 1.88 |
| | f. | Dextroamphetamine sulfate | 3.33 | 1.88 |
| | | (as base) | | |
| | g. | Sodium Polystyrene Sulfonate, | 13.34 | 7.52 |
| | | USP (Amberlite IRP69) (dried | | |
| | | basis) | | |
| | h. | Purified Water | — | Q.S |

| | | | |
|---|---|---|---|
| 3. | Purified Water^{&} | — | Q.S |
| | Total Amount (Solids) | 100.0 | 56.40 mg |
| 4. | Citric acid anhydrous | 0.03 | 4.7 |
| 5. | Propylene glycol | 3.60 | 577.7 |
| 6. | Methylparaben | 0.0375 | 6.0 |
| 7. | Propylparaben | 0.0125 | 2.0 |
| 8. | Xanthan gum | 0.72 | 115.7 |
| 9. | Corn oil | 0.2 | 32.0 |
| 10. | Sorbitol solution non-crystallizing | 25.0 | 4012.7 |
| | (Neosorb) 70% w/w | | |
| 11. | Orange flavor | 0.20 | 32.0 |
| 12. | FDC Yellow # 6 | 0.20 | 32.0 |
| 13. | Sucralose | 0.03 | 4.7 |
| 14. | Purified Water | Q.S to | Q.S to |
| | | 100% | 16050 g |
| | Total Amount (suspension base) | 100.0 | 16.05 g = |
| | | | 15 mL |

^{&}removed during processing;
@ - density of suspension - 1.07 g/cc

### Brief Description of Manufacturing Process:

Drug-Precursor Complex

The precursor resin is added into a known amount of purified water. Mixed at low speed for approximately 2 hours to activate the complexation sites of the resin. Amphetamine salts are then added into the above mixture, stirred for NLT 4 hours to allow complexation. The mixture is then centrifuged, and the supernatant liquid is collected separately. The wet resin is washed with purified water twice before it is placed in the oven for drying. The dried Drug-Precursor resin complex is screened through 20 mesh and 60 mesh to remove the fines and agglomerates.
Drug-Resin Complex

The Amberlite IRP69 resin is added into a known amount of purified water. Mixed at low speed for approximately 2 hours to activate the complexation sites of the resin. At the end of activation, the water is removed through centrifuging. The Supernatant liquid collected from Drug-Precursor resin complexation process is then added into the activated resin and stirred for NLT 4 hours to allow complexation of the resin with the drug present in the supernatant liquid. The mixture is then centrifuged to separate the drug-resin complex. The wet resin is washed with purified water twice before it is placed in the oven for drying. The dried drug-resin complex is screened through 20 mesh to remove any agglomerates.

### Final Product

Propylene glycol is placed in a suitable container, heated to 50-55 ° C. The parabens are then added and mixed until dissolved. Xanthan gum is added to the above mixture, and stirred for 2 hours. In another container, anhydrous citric acid is added into known amount of purified water and mixed until dissolved. The Propylene glycol mixture from container 1 is then added into the aqueous citric acid solution and mixed for 4-5 hours, to allow xanthan gum to hydrate thoroughly. Corn oil, flavor, color, sucralose are added to the mixture one after another, with adequate mixing time after each addition. Sorbitol solution is then added to the above mixture, stirred thoroughly. The solution is then homogenized for 15 minutes. The required amount of Drug-precursor complex and Drug-Resin Complex are added to the mixture and stirred for 1 hour to obtain the final suspension.

### Example 2

### Composition (80: 20% of ER Coated Drug-Precursor Complex: Drug-Resin Complex)

| | | | | Amount/ |
|---|---|---|---|---|
| Sr. | | | Amount | Unit, |
| No. | Ingredients | | (%) | (mg) |
| 1 | Drug - Precursor Complex | | — | 45.12 |
| | a. | Amphetamine sulfate (as base) | 11.50 | 7.52 |
| | b. | Dextroamphetamine sulfate | 11.50 | 7.52 |
| | | (as base) | | |
| | c. | Sodium Polystyrene Sulfonate | 45.98 | 30.08 |
| | | (Amberlite IR69F) (dried basis) | | |
| | d. | Purified Water^{&} | — | Q.S |
| 2. | ER Coated Drug - Precursor Complex^{$\hat{ }} | | — | 54.14 |
| | — | Drug - Precursor Complex | — | 45.12 |
| | e. | Opadry EC (Ethylcellulose mix)^{∞} | 10.61 | 6.94 |
| | f. | Talc | 3.18 | 2.08 |
| | g. | Ethanol 95%^{&} | — | Q.S |
| | h. | Purified Water^{&} | — | Q.S |
| 3. | Drug - Resin Complex^{#} | | — | 11.28 |
| | i. | Amphetamine sulfate (as base) | 2.87 | 1.88 |
| | j. | Dextroamphetamine sulfate | 2.87 | 1.88 |
| | | (as base) | | |
| | k. | Sodium Polystyrene Sulfonate, USP | 11.49 | 7.52 |
| | | (Amberlite IRP69) (dried basis) | | |
| | l. | Purified Water | — | Q.S |

| | | | |
|---|---|---|---|
| 4. | Purified Water^{&} | — | Q.S |
| | Total Amount (Solids) | 100.0 | 65.42 |
| 5. | Citric acid anhydrous | 0.03 | 4.7 |
| 6. | Propylene glycol | 3.60 | 577.7 |
| 7. | Methylparaben | 0.0375 | 6.0 |
| 8. | Propylparaben | 0.0125 | 2.0 |
| 9. | Xanthan gum | 0.72 | 115.7 |
| 10. | Corn oil | 0.2 | 32.0 |
| 11. | Sorbitol solution non-crystallizing | 25.0 | 4012.7 |
| | (Neosorb) 70% w/w | | |
| 12. | Orange flavor | 0.20 | 32.0 |
| 13. | FDC Yellow # 6 | 0.20 | 32.0 |
| 14. | Sucralose | 0.03 | 4.7 |
| 15. | Purified Water | Q.S to | Q.S to |
| | | 100% | 16050 |
| | Total Amount (suspension base) | 100.0 | 16.05 g = |
| | | | 15 mL |

^{&}removed during processing;
^{\hat{ }}15% w/w ER Coating;
@ - density of suspension -1.07 g/cc

### Brief Description of Manufacturing Process

Drug-Precursor Complex

The precursor resin is added into a known amount of purified water. Mixed at low speed for approximately 2 hours to activate the complexation sites of the resin. Amphetamine salts are then added into the above mixture, stirred for NLT 4 hours to allow complexation. The mixture is then centrifuged, and the supernatant liquid is collected separately. The wet resin is washed with purified water twice before it is placed in the oven for drying. The dried Drug-Precursor resin complex is screened through 20 mesh and 60 mesh to remove the fines and agglomerates.

### Coated Drug-Precursor Complex

Ethylcellulose coating dispersion is prepared by dispersing Opadry EC in ethanol-water mixture. Talc solution is homogenized, then added to the above to obtain final dispersion. The drug-precursor complex is coated with ethylcellulose dispersion in fluid bed processor using suitable Wurster column. Weight gain of 15% w/w is targeted.

### Drug-Resin Complex

The Amberlite IRP69 resin is added into a known amount of purified water. Mixed at low speed for approximately 2 hours to activate the complexation sites of the resin. At the end of activation, the water is removed through centrifuging. The Supernatant liquid collected from Drug-Precursor resin complexation process is then added into the activated resin and stirred for NLT 4 hours to allow complexation of the resin with the drug present in the supernatant liquid. The mixture is then centrifuged to separate the drug-resin complex. The wet resin is washed with purified water twice before it is placed in the oven for drying. The dried drug-resin complex is screened through 20 mesh to remove any agglomerates.

### Final Product

Propylene glycol is placed in a suitable container, heated to 50-55 ° C. The parabens are then added and mixed until dissolved. Xanthan gum is added to the above mixture, and stirred for 2 hours. In another container, anhydrous citric acid is added into known amount of purified water and mixed until dissolved. The Propylene glycol mixture from container 1 is then added into the aqueous citric acid solution and mixed for 4-5 hours, to allow xanthan gum to hydrate thoroughly. Corn oil, flavor, color, sucralose are added to the mixture one after another, with adequate mixing time after each addition. Sorbitol solution is then added to the above mixture, stirred thoroughly. The solution is then homogenized for 15 minutes. The required amount of Coated Drug-precursor complex and Drug-Resin Complex are added to the mixture and stirred for 1 hour to obtain the final suspension.

1. A pharmaceutical composition comprising a mixture of dextro- and levo-amphetamines complexed with ion-exchange resin particles to form drug resin particles; wherein the composition comprises two drug-resin complexes; (a) mixture of dextro- and levo-amphetamines-resin complex providing for immediate release; and (b) mixture of dextro- and levo-amphetamines-precursor resin complex covered with an extended release coating; wherein 1% to 30% by weight of the amphetamines are present in (a) drug-resin complex and 70% to 99% by weight of the amphetamines are present in (b) drug resin complex.

2. The pharmaceutical composition according to claim 1, wherein extended release coating comprising water permeable and film forming polymer.

3. The pharmaceutical composition according to claim 2, wherein extended release coating comprising ethyl cellulose.

4. The pharmaceutical composition according to claim 3, wherein (a) drug resin complex comprises mixture of dextro- and levo-amphetamines and sodium polystyrene sulfonate having particle size in the range of 47 to 149 microns.

5. The pharmaceutical composition according to claim 4, wherein (b) drug resin complex comprises mixture of dextro- and levo-amphetamines and sodium polystyrene sulfonate beads having particle size in the range of 250 to 600 microns.

6. The pharmaceutical composition according to claim 5, wherein the (a) and (b) drug resin complexes can be incorporated into an aqueous based orally acceptable pharmaceutical carrier.

7. The pharmaceutical composition according to claim 5, further comprises sweeteners, flavoring agents, colorants, acidulants, antioxidants or preservatives thereof.

8. The pharmaceutical composition according to claim 7, is liquid composition.

9. The pharmaceutical composition according to claim 8, administered once daily to the patient.

10. The pharmaceutical composition according to claim 1, can be administered to the patients suffering from various conditions such as Attention-Deficit Disorder or ADHD, fatigue, obesity or imparting alertness.
